(19) United States
(12) Patent Application Publication
(10) Pub. No.: US 2025/0101377 A1
(43) Pub. Date: Mar. 27, 2025

(54) METHODS AND COMPOSITIONS OF TREATING SPINAL CORD INJURIES

(71) Applicant: City University of Hong Kong, Hong Kong (HK)

(72) Inventor: Aijia LIU, Hong Kong (HK)

(21) Appl. No.: 18/472,200

(22) Filed: Sep. 21, 2023

(51) Int. Cl. C12N 5/0797; A61P 25/00

(52) U.S. Cl. CPC C12N 5/0623; A61P 25/00

(57) ABSTRACT

A method for treating spinal cord injuries involves the administration of a genetically modified neural progenitor cell (NPC). This genetically engineered NPC is designed to exhibit an enhanced sonic hedgehog (SHH) signaling pathway. This method aims to provide a therapeutic strategy for addressing spinal cord injuries by leveraging the augmented SHH signaling pathway in the administered cells.

### FIELD OF THE INVENTION

The present invention generally relates to the medical field. More specifically the present invention relates to methods and compositions for treating spinal cord injuries.

### REFERENCE TO SEQUENCE DISCLOSURE

The sequence listing file under the file name “P2862US00_SEQ.xml” submitted in ST.26 XML file format with a file size of 3.28 KB created on Sep. 11, 2023 and filed on Sep. 21, 2023 is incorporated herein by reference.

### BACKGROUND OF THE INVENTION

Traumatic spinal cord injury (SCI) represents a profoundly debilitating condition characterized by the progressive loss of neurons and oligodendrocytes. This results in irreversible axonal damage, impaired locomotion, and compromised somatosensory function. Regrettably, existing clinical management and treatment approaches for SCI patients with enduring disabilities remain ineffective. After the injury, the neurons within the adult mammalian central nervous system (CNS) possess limited regenerative potential, while the extrinsic injury environment lacks the necessary neurotrophic support required for restoring spinal composition and architectural integrity.

Exogenous grafts with therapeutic cell populations have shown promise to compensate for the loss of the damaged nervous system and improve functional recovery after SCI. In most studies, neural progenitor cells (NPCs) embedded in a fibrin-thrombin matrix containing neurotrophic factors are required to enable the survival and differentiation of grafts at the injury sites. However, the therapeutic effects are often limited by the pro-longed maturation process of hNPCs and the hostile niche that promotes grafts to differentiate into astrocytes instead of neurons. Additionally, SCI leads to the formation of glial scar, which creates a barrier-like structure that prevents regenerated axons from penetrating and projecting across the lesion site.

Therefore, the art still seeks for a genetically modified hNPCs with enhanced survival and neuronal differentiation properties and the capability to modulate the host environment as an effective approach for treating traumatic SCI, and the present invention addresses this need.

### SUMMARY OF THE INVENTION

It is an objective of the present invention to provide methods, compositions, or usages to solve the aforementioned technical problems.

In accordance with a first aspect of the present invention, a method for treating spinal cord injury, more particularly, the method includes administering a genetically engineered neural progenitor cell (NPC) is provided, in which the genetically engineered NPC's sonic hedgehog (SHH) signaling pathway is upregulated intrinsically and engineered NPCs also produce SHH protein extrinsically.

In accordance with one embodiment of the present invention, the upregulation of SHH signaling pathway is achieved by suppressing a negative regulator of SHH signaling pathway.

In accordance with another embodiment of the present invention, the negative regulator is suppressor of fused homolog (SUFU).

In accordance with one embodiment of the present invention, the administration is grafting the genetically engineered NPC directly into a lesion site of a subject in need thereof.

In accordance with one embodiment of the present invention, the upregulated SHH signaling pathway reduces apoptosis and enhances the survival and differentiation of host neurons and engineered NPCs at the lesion site.

In accordance with one embodiment of the present invention, the genetically engineered NPC reduces suppressive barriers derived from glial scar around the lesion sites.

In accordance with one embodiment of the present invention, the administration of the genetically engineered NPC is combined with a physical therapy regimen to enhance functional recovery.

In accordance with a second aspect of the present invention, a composition including a genetically engineered NPC, in which the SHH signaling pathway is upregulated, for treating spinal cord injury in a subject in need thereof, and a pharmaceutically acceptable addition is provided.

In accordance with one embodiment of the present invention, the upregulation of SHH signaling pathway is achieved by suppressing a negative regulator of SHH signaling pathway.

In accordance with another embodiment of the present invention, the negative regulator is SUFU.

In accordance with one embodiment of the present invention, the pharmaceutically acceptable addition includes an excipient, a stability additive, a carrier, a diluent, and a solubilizer.

In accordance with one embodiment of the present invention, the pharmaceutically acceptable additive further includes a neurotrophic factor.

In accordance with one embodiment of the present invention, the composition further includes a sustained release system to provide prolonged exposure of the genetically engineered NPC at the lesion site.

In accordance with one embodiment of the present invention, the composition is formulated for direct delivery to a lesion site of the subject.

In accordance with one embodiment of the present invention, the composition is formulated as an injectable gel or scaffold for controlled delivery to the lesion site.

In accordance with a third aspect of the present invention, a usage of a composition having a genetically engineered NPC for treating spinal cord injury in a subject in need thereof is provided, more particularly, the genetically engineered NPC's SHH signaling pathway is upregulated.

In accordance with one embodiment of the present invention, the upregulation of SHH signaling pathway is achieved by suppressing a negative regulator of the SHH signaling pathway.

In accordance with one embodiment of the present invention, the negative regulator is SUFU.

In accordance with one embodiment of the present invention, the genetically engineered NPC's upregulated SHH signaling pathway promotes remyelination of damaged axons.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIGS. 1A-1H depict the molecular characterization of SUFU-KD hNPCs, in which FIG. 1A shows the mRNA and protein expression levels of SUFU of hNPCs analyzed by immunoblotting and qPCR, FIG. 1B depicts the qPCR analysis of markers associated with SHH effectors, FIG. 1C exhibits the representative bright-field images showing the formation and renewal of neurospheres, FIG. 1D shows the quantification of neurosphere number, FIG. 1E shows the quantification of neurosphere size, FIG. 1F depicts the representative immunofluorescence imaging for OLIG2, SOX2, HuC/D, PAX6, and NKX6.1 in hNPCs, FIG. 1G shows the quantification of the immunofluorescence imaging for OLIG2, SOX2, HuC/D, PAX6, and NKX6.1 in hNPCs, and FIG. 1H shows qPCR analysis of dorsal-ventral markers (PAX6,OLIG2,PAX7), neural genes (SOX2/1, Nestin) and neuronal markers (MPA2, TUJ1, ISL1/2, and HB9) of hNPCs;

FIGS. 2A-2I depicts that hNPCs with SUFU inhibition exert cell intrinsic differentiation capacity in generating neurons and oligodendrocytes, in which FIG. 2A shows the representative immunofluorescence of neurofilament (NF) from scramble and SUFU-KD hNPCs at 7 days differentiation in the absence of neurotrophic factors while dotted line indicates the border of neurosphere, FIG. 2B shows the quantification of relative intensity of axon outgrowth in neurospheres, FIG. 2C shows the representative immunofluorescence images for MAP2, and ISL1/2 hNPCs at 7 days differentiation, FIG. 2D shows the percentage of ISL1/2-positive cells in hNPCs after 14 days differentiation, FIG. 2E depicts the representative immunofluorescence images for ChAT, HB9, MAP2, and synaptophysin (SYN) of hNPCs at 21 days differentiation with white boxes showing magnified view with indicated markers, FIG. 2F is the quantification of the expression of ChAT, HB9, MAP2, and synaptophysin (SYN) of hNPCs at 21 days differentiation, FIG. 2G shows the qPCR analysis of HH effectors (SUFU,GLI1,PATCH1), neural genes (SOX2 and PAX6) and neuronal markers (MPA2, TUJ1, ISL1/2, ChAT, and HB9) at 21 days differentiation, FIG. 2H shows the representative immunofluorescence images for GFAP, NG2 and SOX10 after 3 weeks differentiation in hNPCs with white boxes showing magnified view with indicated markers, and FIG. 2I depicts the quantification of GFAP, NG2 and SOX10 expression in hNPCs after 3 weeks differentiation;

FIGS. 3A-3H depict non-cell autonomous effects of SUFU-KD cells, in which FIG. 3A depicts the representative immunofluorescence images showing increased SHH production from SUFU-KD hNPCs, FIG. 3B shows the quantification of SHH production from SUFU-KD hNPCs, FIG. 3C shows the representative immunofluorescence images for caspase 3, neuronal marker MAP2 and nuclei marker DAPI scramble and SUFU-KD hNPCs after 14 days treatment of homogenate (100 μg/ml) from the injured spinal cord, FIG. 3D shows the quantification of caspase 3+ cells from FIG. 3C, FIG. 3E depicts the schematic diagram showing co-culture of scramble and SUFU-KD NSP with and without GFP, FIG. 3F shows the representative immunofluorescence images for caspase 3, HuC/D and GFP in co-cultures of Scramble+Scramble (GFP), Scramble+SUFU-KD1 (GFP), and Scramble+SUFU-KD2 (GFP) with white boxes showing a magnified view with indicated markers, FIG. 3G shows the percentage of caspase 3+ cells in GFP positive or GFP negative cells in co-cultures of Scramble+Scramble (GFP), Scramble+SUFU-KD1 (GFP), and scramble+SUFU-KD2 (GFP), FIG. 3H shows the percentage of HuC/D in total GFP cells in co-cultures of Scramble+Scramble (GFP), Scramble+SUFU-KD1 (GFP), and Scramble+SUFU-KD2 (GFP);

FIGS. 4A-4G depict that SUFU-KD grafts display efficient integration and modulate injured niche in the SCI model, in which FIG. 4A shows the representative images of caspase 3 positive cells in the injured spinal cord without grafting or with scramble and SUFU-KD1 grafts at 1 month (1M) post-graft with white boxes showing a magnified view with indicated markers, FIG. 4B shows the quantification of the caspase 3 positive cells in FIG. 4A, FIG. 4C depicts the representative immunofluorescence images for GFP, MAP2 (red), and GFAP (Blue) in sagittal sections with scramble and SUFU KD1 grafts at 1 month (1M) post-graft, and the cystic lesion cavity (LC) is formed with surrounding dense GFAP immunoreactivity (blue), whereas SUFU-KD1 grafts (GFP-positive) crossed GFAP barriers and dot line indicates the dense astrocytic glia marked by GFAP, FIG. 4D shows the representative immunofluorescence images for GFP, CSPG (red), and DAPI (Blue) in spinal cord sagittal sections grafted with scramble and SUFU-KD1 hNPCs at 1 month post-graft (1M), and the cystic lesion cavity (LC) is formed with surrounding dense CSPG immunoreactivity (red) and SUFU-KD1 grafts with GFP expression attenuate CSPG graft/host interface, FIG. 4E shows the fluorescence intensity analysis of CSPG surrounding the lesion cavity, FIG. 4F shows that GFP and Neurofilament (NF) immunolabeling in spinal cord sagittal sections reveal GFP-expressing SUFU KD1 grafts at injured sites generated robust axons extending into the host spinal cord caudally after 2 months post-graft (2M) with a-a″ and b-b″ indicating higher magnification of NF-positive fibers in the graft at different regions from rostral to caudal, and FIG. 4G shows the quantification of axon intercepts at specific distances from graft-host border in the injured cord grafted with scramble and SUFU KD1 hNPCs;

FIGS. 5A-5H depict that SUFU-KD hNPC grafts promote beneficial differentiation intrinsically and extrinsically in the SCI model, in which FIG. 5A shows the representative immunofluorescence images for GFP, GlyT2 (red), and ISLET1/2 (Blue) in sagittal sections of injured spinal cord with scramble and SUFU-KD1 grafts at 2 month (1M) post-graft with empty arrows showing the indicated markers expression in GFP positive cells, white arrows showing the indicated markers in GFP negative cells and white boxes showing a zoomed-in view of the co-localization of indicated markers, FIG. 5B shows the quantification of the percentage of GlyT2 and ISLET1/2 in grafts or non-grafts cells of FIG. 5A, FIG. 5C depict the representative immunofluorescence images for GFP, GABA (red) and CaMKII (cyan) in sagittal sections with scramble and SUFU-KD1 grafts at 2 month (1M) post-graft with empty arrows showing the indicated markers expression in GFP positive cells, white arrows showing the indicated markers in GFP negative cells, and white boxes showing a zoomed-in view of the co-localization of indicated markers, FIG. 5D shows the quantification of the percentage of GABA in grafts or non-grafts cells of FIG. 5C, FIG. 5E shows the quantification of the percentage of CaMKII in grafts or non-grafts cells of FIG. 5C, FIG. 5F shows the representative immunofluorescence images for GFP, SOX10 (red) and HNFEL(Blue) in sagittal sections with scramble and SUFU-KD1 grafts at 2 month (1M) post-graft with white boxes showing a zoomed-in view of the co-localization of indicated markers, FIG. 5G shows the quantification of the percentage of SOX10-in GFP-positive or negative cells in the injured cord grafted with scramble and SUFU-KD1 hNPCs, and

FIG. 5H shows GFP-positive grafts from scramble and SOX9 KD immunolabeled with HNEFL and re-myelination marker (MBP, red) at 3 months post-graft with insets showing the distribution of GFP-positive grafts in the injury site and boxes with dotted line showing a zoomed-in view with indicated markers;

FIGS. 6A-6F depict the graft-initiated trans-synaptic AAV virus antegrade labeling of host connectivity, in which FIG. 6A depicts the sagittal section showing antegrade, trans-synaptically traced host mCherry-expressing cells in the injured spinal cord with Scramble and SUFU-KD graft with inset image showing injection sites in brain region and a-a″ “′ and b-b”′ indicating high-magnification view of boxed area, FIG. 6B depicts the representative immunofluorescence images showing mCherry+GFP+ trans-synaptically connection from motor cortex to the grafts in the lesion sites, FIG. 6C depicts the quantification of the proportion of mCherry labelled cells/axons in scramble of SUFU-NPC grafts, normalized to the total number of mCherry labelled axons located 0.5 mm rostral to the lesion site, FIG. 6D depicts the transverse sections labelled for mCherry and GFP at L2 host spinal cord levels, showing that host neurons monosynaptically connected to grafts are detected over long lengths of the rat spinal cord with white arrowheads indicating mCherry traced motoneurons (ChAT+) in SUFU-KD grafts, FIG. 6E shows the quantification of mCherry traced neurons in L1-L2 in scramble and SUFU-KD grafts, and FIG. 6F shows the triple labeling for GFP, 5-HT, and human synapsin (hSYN) revealing colocalization of regenerating 5-HT axon terminals with hSYN, suggesting synaptic connectivity;

FIGS. 7A-7D depict the significant functional improvement after transplantation of SUFU-KD1 hNPC grafts into contusive SCI, in which FIG. 7A demonstrates the BBB scores of lesion control, and pre- and post-grafting with scramble and SUFU-KD1 hNPCs, FIG. 7B shows the grid walk quantitative analysis measured as a percentage of hind limb placement, FIG. 7C exhibits the foot fault score analysis of hind limb measured by rating scale for foot placement in the skilled ladder rung walking test, and FIG. 7D shows the quantification of stride length in sham, SCI(lesion control) and SCI rats with scramble and SUFU-KD1 grafts;

FIGS. 8A-8G depict the generation and characterization of SUFU knockdown human pluripotent stem cell lines, in which FIG. 8A display a schematic showing steps involved in differentiation of human pluripotent stem cells (hPSCs) to human neural stem cells (hNSCs), FIG. 8B shows the qRT-PCR analysis results, FIG. 8C depicts the western blot analysis of SUFU proteins in different groups, FIG. 8D shows the mRNA levels of SUFU in different groups, FIG. 8E shows the mRNA levels of GLI1, GLI2, HIP1 and PATCH1 in different groups, FIG. 8F demonstrates the mRNA levels of pluripotent stem cell markers in different groups, and FIG. 8G displays the FACS analysis outcomes of pluripotent markers, apoptotic marker and EdU incorporation assays in different groups; and

FIG. 9 depicts the AA virus antegrade labeling of host connectivity in SCI lesion control.

### DETAILED DESCRIPTION

In the following description, methods, compositions, and/or usages of treating spinal cord injury and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Traumatic spinal cord injury, caused by compression, contusion or laceration, often leads to the rapid necrotic death and loss of neurons and glia. This is followed by the activation of astrocytes and inflammatory responses, resulting in widespread changes in extracellular matrix molecules in the microenvironment. Consequently, the complex post-injury milieu and the failure of nerve tissue regeneration following SCI causes varying degrees of paralysis, depending on the level and severity of the injury. Stem cell transplantation, especially human NPCs derived from human pluripotent stem cells (hPSCs), offers a potential strategy for the treatment of SCI. These grafts have been shown to be a promising cell source for repairing injured spinal cord because of their competency to generate neurons for restoration of neuronal circuitry and oligodendrocytes for remyelination of spared axons. However, the functional outcome of grafted hNPCs that largely depends on the survival, differentiation potency, integration capacity and axial identity is hampered by the hostile micro-environment at and around the lesion sites. Additionally, the amount of oligodendrocytes generated is often insufficient for remyelination of spared axons. Previous studies have shown that activation of signalling pathway, such as SHH and GDNF in the post-injury microenvironment can modulate the niche and enhance the viability and differentiation potency of the grafted cells. Nevertheless, the beneficial effects of these growth factors on the grafted cells or the niche might be transient and limited by the prolonged differentiation/maturation process of human cells.

As used herein, the term “suppressing” means completely eliminated (“silencing”) or substantially reducing the impact (“knocking down”) a gene and/or a signaling pathway.

In accordance with a first aspect of the present invention, the present invention provides a method for the treatment of spinal cord injuries. Spinal cord injuries lead to a gradual loss of neurons and oligodendrocytes, resulting in irreversible damage to axons and a subsequent decline in both locomotion and somatosensory function. Regrettably, existing treatment options for individuals with spinal cord injuries remain insufficient, underscoring the need for innovative therapeutic strategies to address this pressing medical concern.

The core of this method revolves around harnessing the potential of genetically engineered neural progenitor cells (NPCs) as a means to facilitate spinal cord recovery. NPCs have demonstrated a remarkable ability to contribute to neural tissue restoration and functional improvement. What sets this approach apart is the intentional manipulation of the sonic hedgehog (SHH) signaling pathway within these genetically modified NPCs.

Central to the method is the precise modulation of the SHH signaling pathway. This modulation involves the targeted suppression of a critical negative regulator of the SHH pathway, known as suppressor of fused homolog (SUFU). Through this strategic regulatory control, the SHH signaling pathway is amplified, leading to a cascade of cellular responses that hold significant promise for spinal cord repair and regeneration.

The sonic hedgehog (Shh) signaling pathway plays essential roles in neurodevelopment, contributing to renewal and survival of NPCs, neuronal differentiation, axon growth and cell fate specification of motoneurons and oligodendrocytes. SHH activation using small molecules (e.g., purmorphamine) is required for generating therapeutic cell types from human pluripotent stem cells (hPSCs) for SCI grafts, including NPCs, motoneurons, and oligodendrocytes. Moreover, the transplantation of both motoneurons and oligodendrocytes into a rat SCI model led to better locomotor recovery than transplanting individual cell types. In SCI, SHH is often downregulated and suppressed by reactive astrocytes around the lesion site. Recent studies have revealed neuroprotective and regenerative features of administrating SHH after CNS injuries, which enhances axonal outgrowth, counters apoptosis, and modulates the microenvironment via promoting neurotrophic factor secretion, suggesting that genetic manipulation of SHH signaling components can be an effective strategy in treating SCI.

Suppressor of fused (SUFU) is a negative regulator of SHH signaling but exhibits a distinct mode of action compared to SHH inhibitors. In the absence of SHH, SUFU forms inhibitory complexes with Gli1-3 transcriptional effectors of SHH signalling in the cytoplasm and promotes the proteolytic cleavage of the full-length Gli3 activator (Gli3A190) to form the Gli3 repressor (Gli3R83). Dissociating the SUFU-GL1 complex following SHH stimulation allows the GL1 proteins to be translocated into the nucleus and converted to transcriptional activators. Previous studies show that Sufu−/− embryos exhibited expanded Shh production in the neural tube and increased expression of Olig2, a maker for the progenitor of both motoneurons and oligodendrocytes that is normally induced by Shh signalling20. In vertebrate development, specific deletion of Sufu in neural/neural crest progenitors resulted in the early onset of neuronal or glial differentiation, depending on the cellular context 21-24. These findings raise the possibility that inactivating SUFU in hNPCs could exert beneficial effects on the injured spinal cord via SHH activation.

In the present method, the engineered human pluripotent stem cell (hPSC)-derived NPCs with SUFU inhibition exhibits enhanced survival, neuronal differentiation with extensive neurites outgrowth, and ability to generated oligodendrocytes, counteracting the harmful effects from the injured niche in vitro and in vivo. Importantly, these modified NPCs can exert non-cell autonomous effects by secreting SHH proteins, enhancing the survival and neurogenesis of non-modified NPCs in vitro. Upon grafting, NPCs with SUFU inhibition modulate the injured microenvironment and host cells, which dramatically reduces suppressive barriers derived from glial scar around lesion sites. Notably, a significant increase is observed in the number of mature neurons with different subtypes around SUFU-knockdown grafts, indicating the role of modified grafts in preventing progressive apoptosis/loss or/and promoting differentiation of host cells. The combinational beneficial effects of NPCs with SUFU inhibition result in more effective integration without requiring a supportive matrix, enhancing the repair of spinal cord lesions in a post-injury hostile milieu and improving locomotor function.

The method involves the careful grafting of genetically engineered NPCs directly into the specific lesion site of the spinal cord injury. This localized administration strategy ensures that the modified cells can exert their effects precisely where they are needed most. By targeting the lesion site, the method maximizes the potential for positive treatment outcomes.

A notable outcome of enhancing the SHH signaling pathway is the impact on host neurons situated at the lesion site. The heightened SHH signaling environment created by the genetically engineered NPCs provides a conducive environment for the survival and differentiation of these neurons, restoring the integrity of neural networks and fostering functional recovery.

Furthermore, this method can be synergistically integrated with tailored physical therapy regimens to optimize the potential for functional recovery. By combining the regenerative properties of the modified NPCs with targeted physical interventions, the treatment strategy leverages the plasticity of the neural system, accelerating the recovery process.

In conclusion, the method introduced herein offers an avenue for tackling spinal cord injuries. Through the utilization of genetically engineered NPCs and the strategic manipulation of the SHH signaling pathway, this approach mitigates the impact of spinal cord injuries and restores vital neurological functions. The integration of physical therapy further enhances its therapeutic potential, providing a comprehensive and effective strategy for addressing these challenging conditions.

Furthermore, the present invention provides a composition that holds immense potential within the realm of spinal cord injury treatment. This composition centers around genetically engineered neural progenitor cells (NPCs), meticulously designed to activate the SHH signaling pathway, a critical factor in spinal cord repair and recovery.

The activation of the SHH signaling pathway involves the strategic upregulation of the SHH signaling pathway by targeting its negative regulator. By suppressing this regulator, known as suppressor of fused homolog (SUFU), the SHH signaling pathway activity is enhanced and provides a promising treatment for spinal cord injury.

The composition includes a range of pharmaceutically acceptable additives, including excipients, stability additives, carriers, diluents, and solubilizers. These additives not only enhance the therapeutic efficacy of the composition but also ensure its compatibility with biological systems, facilitating safe and impactful treatment.

In addition, the composition may include neurotrophic factors. This factor, combined with genetically engineered NPCs and heightened SHH signaling pathway activity, creates an environment conducive to neural survival, differentiation, and regeneration.

To prolong the therapeutic impact, the composition may feature a sustained release system. This mechanism ensures a consistent and prolonged presence of genetically engineered NPCs at the lesion site, fostering an environment favorable for spinal cord repair.

The composition's specificity lies in its direct delivery to the lesion site. Tailored for precise administration to the injured region within the spinal cord, this approach optimizes the treatment's efficiency, enhancing its impact on neural recovery.

In one embodiment, the composition's adaptability is showcased through its formulation as an injectable gel or scaffold. This allows for controlled and targeted delivery to the lesion site, enhancing the composition's overall effectiveness.

Moreover, the present invention provides an application of a composition for the treatment of spinal cord injuries.

The application focuses on the utilization of the composition including genetically engineered NPCs. These cells have been meticulously crafted to exhibit an upregulated SHH signaling pathway, a mechanism crucial for spinal cord repair and recovery.

As mentioned above, the core feature of this approach is the strategic manipulation of the SHH signaling pathway by targeting its negative regulator, SUFU, thereby promoting the heightened activity of the SHH signaling pathway.

The application also encompasses the use of this composition to encourage the remyelination of damaged axons. Through the orchestrated activation of the genetically engineered NPC's upregulated SHH signaling pathway, the composition fosters an environment conducive to remyelination. This pivotal function addresses one of the key challenges faced in spinal cord injuries—the restoration of axonal integrity and function.

By employing genetically engineered NPCs to enhance the SHH signaling pathway, the composition creates a microenvironment favorable for spinal cord repair. The targeted suppression of SHH signaling pathway inhibitors further amplifies the therapeutic impact, promoting a multi-faceted approach to treatment.

### EXAMPLES

### Example 1. Generation and Characterization of SUFU Knockdown hNPCs Derived from Human Pluripotent Stem Cells

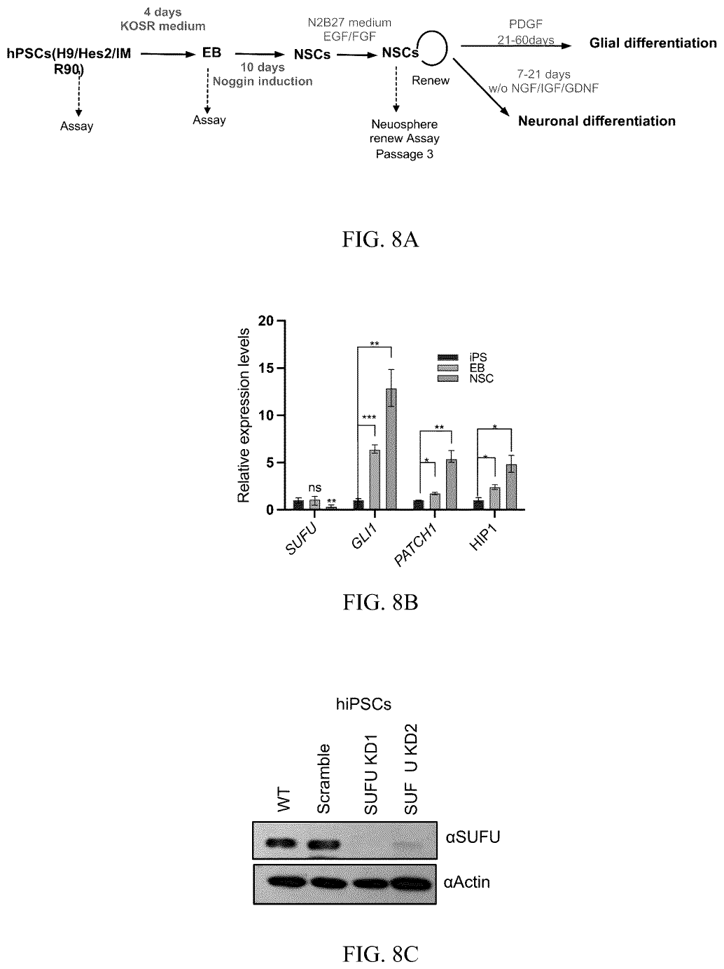

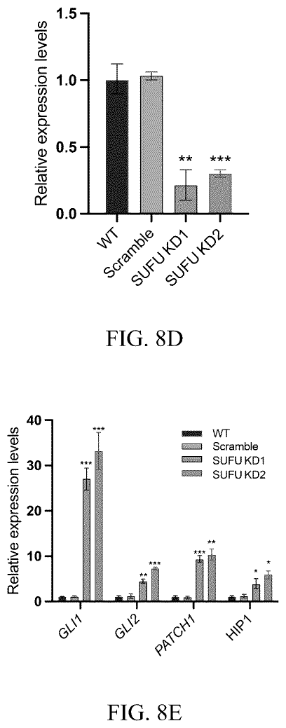

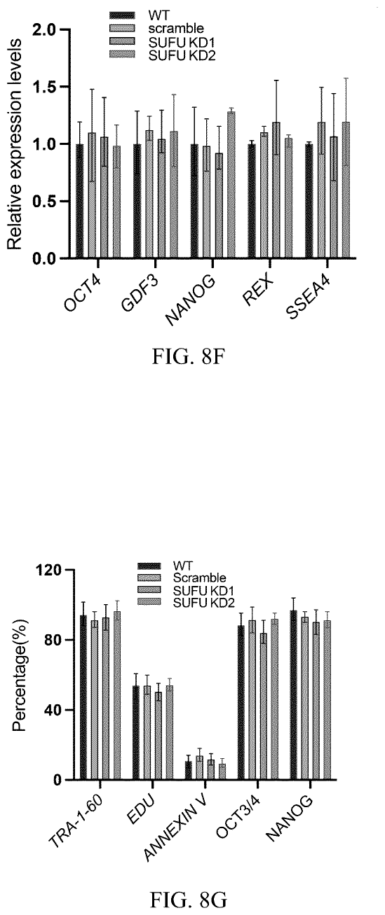

SUFU is a highly conserved negative regulator of the SHH signaling pathway. Studies have shown that deletion or inhibition of SUFU increases SHH activity, promoting the expression of Olig2, a key regulator of motoneuron and oligodendrocytes differentiation, and leading to an early onset of neurogenesis and glial differentiation with transiently enhanced proliferation in the CNS and PNS. It suggests that the inactivation of SUFU triggers intrinsic specification/differentiation program without the need for extrinsic signaling. Hence, it is investigated whether genetic manipulation of SUFU levels in hPSCs-derived NPCs triggers the precocious acquisition of therapeutic cell types to enhance their regenerative potential for treating SCI. To address this issue, an in vitro differentiation protocol is established (FIG. 8A) to examine the transcript levels of SUFU and SHH effectors GLI1, PATCH1, and HIP1 during neural induction from three hPSCs lines (IMR90/Hes2/H9). Briefly, hPSCs (IMR90/Hes2/H9) are provided by WiCell Research Institute (Madison, WI), passaged 33-49. Cells are cultured on Matrigel gel (Corning)-coated plates in mTeSR (Stem Cell Technologies) or E8 medium (Thermo Fisher Scientific, Waltham, MA). Cells are passaged with ResLR (Stem Cell Technologies), washed, and replated at a dilution of 1:5 or 1:10. IMR90 is used for the rest of the studies, although three cell lines display similar results. The qPCR results shows that reduced SUFU expression coincides with upregulated SHH target gene expression, indicating the elevation of SHH signaling in hNPCs during differentiation when compared with hiPSCs and embryoid body (EB) (FIG. 8B). These findings suggest that reduced SUFU expression and activation of SHH signalling coincide with the acquisition of hNPC fate.

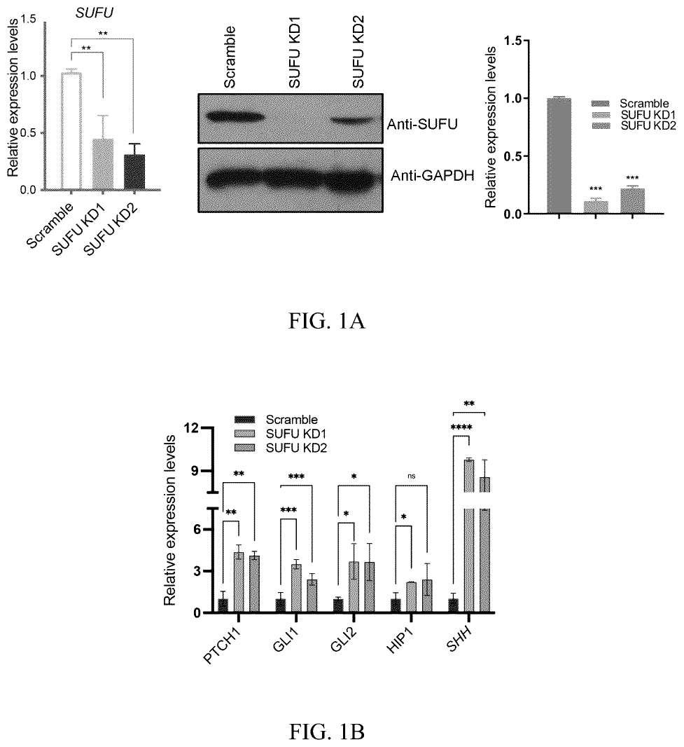

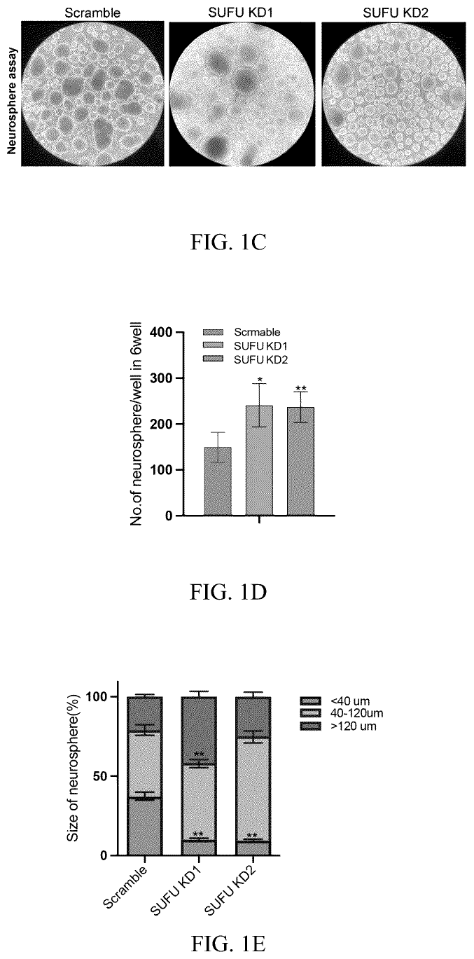

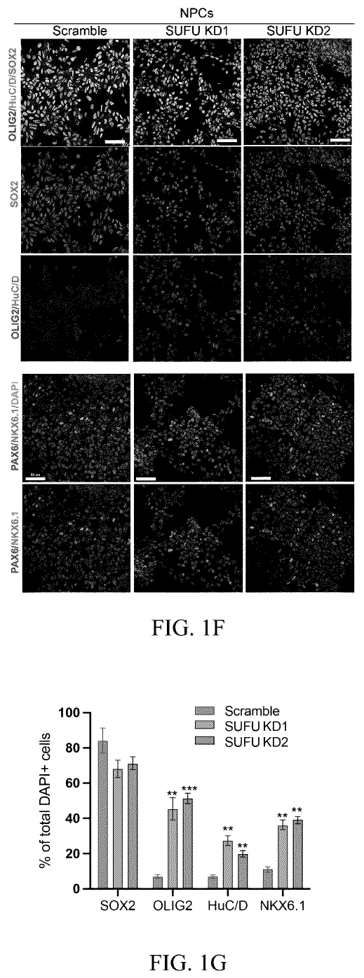

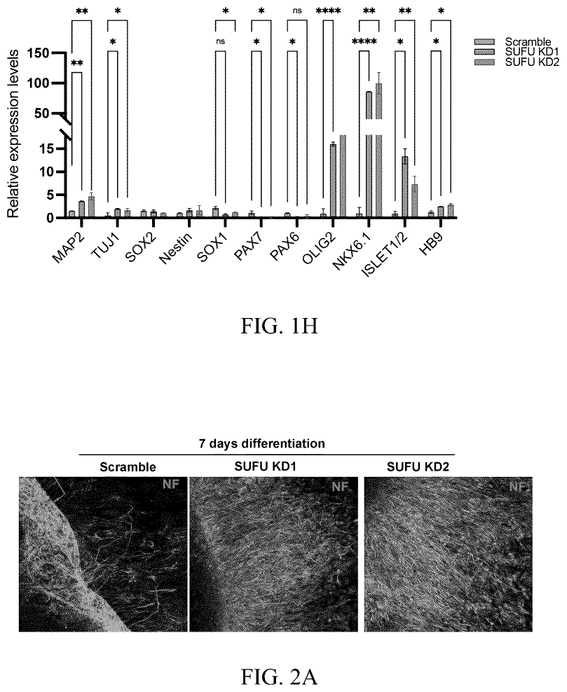

To further explore the link between the reduction of SUFU levels and the specification/differentiation potential of hNPCs, three hPSCs lines (H9, HES2, and IMR90) are infected with two lentiviral-mediated short hairpin RNAs (shRNAs) against the linker region of SUFU (SUFU-KD1 and -KD2) or scramble as control by lentivirus (FIG. 1A and FIG. 8D). Briefly, shRNA against human SUFU-KD1 (SEQ ID No: 1) and SUFU-KD2 (SEQ ID No: 2) are designed using the principles from The RNAi Consortium, and the shRNAs are cloned into lentiviral vector pLKO.1-puro/eGFP and the pLKO.1-TRC control (Addgene plasmid #10879). The human SUFU cDNA is cloned into lentiviral vector pLVX-EF1α-puro (Clontech). To produce lentivirus production, 5×10⁶ 293 T cells are plated in a 100-mm dish and transfected with a lentiviral expression vector, packaging plasmid psPAX.2, and envelope plasmid pMD2.G using in vitro DNA transfection reagent. The cell culture medium containing the lentiviral particles is harvested at 48 and 72 h post-transfection, filtered through a 0.45-μm filter. The lentiviral particles are titrated by lentivirus titration qRT-PCR kit. Next, 3×10⁵ human pluripotent stem cells are infected with quantified lentivirus particles expressing cDNA and/or shRNA and cultured in the presence of 8 μg/mL polybrene for 24 h. After 48 h of transduction, infected cells are screened in presence of 1 μg/mL puromycin. For enrichment of GFP expressing cells for transplantation, GFP-positive hiPSCs or NPCs derived from GFP-hiPSCs are expanded after flow cytometry. The cell suspension is washed in DMEM and filtered through a 50-μm nylon cell strainer. The single-cell suspension is collected by centrifugation and suspended in mTesR or neural maintenance media and then subjected to Fluorescence-activated cell sorting (FACS). Enriched GFP-positive cells are expanded for 3-4 weeks (up to five passages) before grafting or qPCR analysis.

Consistent with the inhibitory role of SUFU in SHH signaling, expressions of SHH and its downstream targets GLI1, GLI2, PATCH1 and HIP1 are significantly upregulated in SUFU KDs hiPSCs compared to their expression levels in scramble and wild-type (WT) hiPSCs (FIGS. 8C-8E). However, SUFU-KD hiPSCs with activated SHH pathway exhibit similar expression levels of pluripotent stem cell markers compared to scramble and WT hPSCs (FIG. 8F). In addition, EdU incorporation and annexin V apoptosis assays show no effects on proliferation and viability in SUFU-KD hiPSCs (FIG. 8G). These results show that the activation of SHH signaling does not affect pluripotency, proliferation, and survival of hPSCs.

Next, the effects of SUFU inhibition in hNPCs are evaluated. Consistently, both SUFU-KD1 and -KD2 hNPCs (passage <5) exhibit significantly reduced SUFU mRNA and protein expressions concomitant with an upregulation of SHH target genes compared to scramble control (FIG. 1A and FIG. 1B). The affection of the reduced SUFU expression levels on the self-renewal capacity of hNPCs is further evaluated using a neurosphere assay in the absence of growth factors, EGF and FGF. Briefly, for the neurosphere assay, NPCs at passage 1 are dissociated into a single-cell suspension at 10⁵/mL and transferred onto 6-well low attachment plates (Corning). Neurospheres are cultured in neural maintenance medium to 3 mL in each well. For secondary neurosphere formation, primary neurospheres are further dissociated into a single-cell suspension after 7 days, and 10⁵/mL of the cell suspension is transferred into 6-well low attachment plates. The results show that the morphology of secondary neurospheres (P2) from SUFU-KDs group exhibit more spherical and transparent multicellular complexes compared to scramble control (FIG. 1C and FIG. 1D). Importantly, SUFU inhibition significantly increases the number of neurospheres and reduces the formation of small-sized (<40 μm) neurospheres (FIG. 1D and FIG. 1E). The impacts of SUFU inhibition on the fate of hNPCs are next examined. Compared with scramble group, SUFU-KD1 and -KD2 hNPCs have much higher proportions of cells expressing ventral neural progenitor marker NKX6.1, motor neuron/oligodendrocytes progenitor marker OLIG2, and pro-neuronal marker HuC/D with subtle reduction of SOX2 that may be due to accelerated neurogenesis (FIG. 1F and FIG. 1G). Consistently, qPCR analysis reveals significant upregulated ventral neural patterning genes (NXK2.2, NKX6.1, and OLIG1/2) and motor neuronal genes (Tuj1, HB9, and ISLET1/2) but downregulated neural plate border markers PAX7 (FIG. 1H). These results indicate that SUFU-KD NPCs exhibit enhanced activation of SHH signaling with the acquisition of pro-neurogenic potency bias toward motor neurons and oligodendrocyte lineages.

### Example 2. SUFU Inhibition Triggers a Spontaneous Differentiation Program to Promote the Formation of Motoneurons and Oligodendrocytes

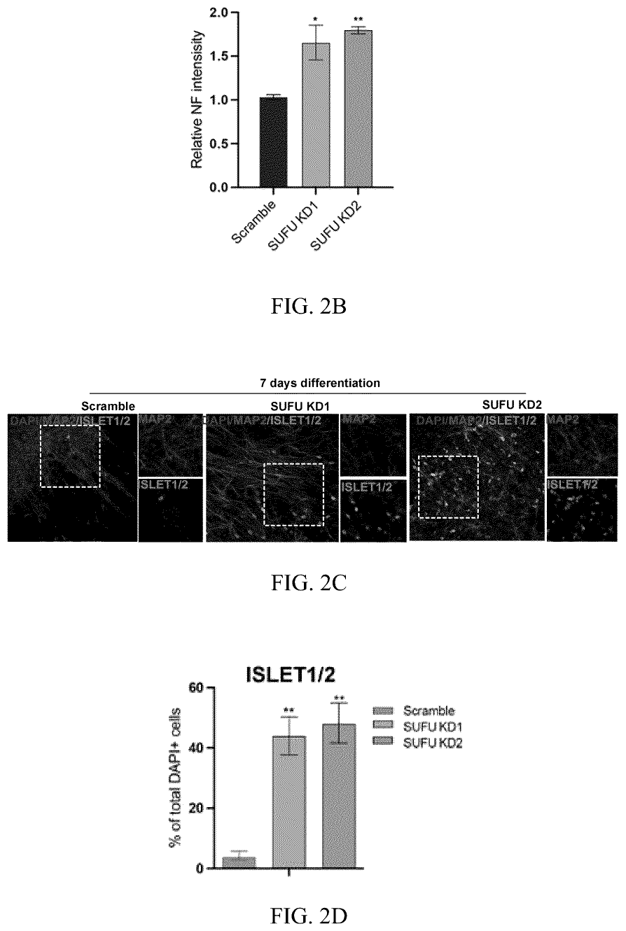

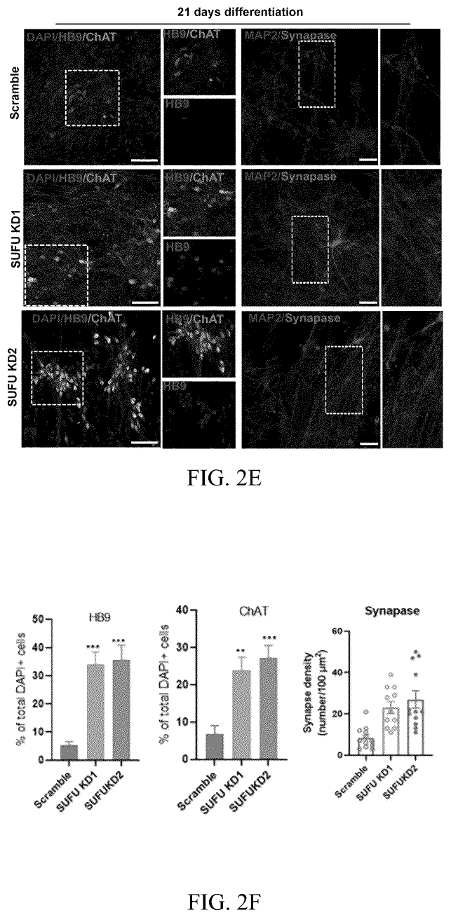

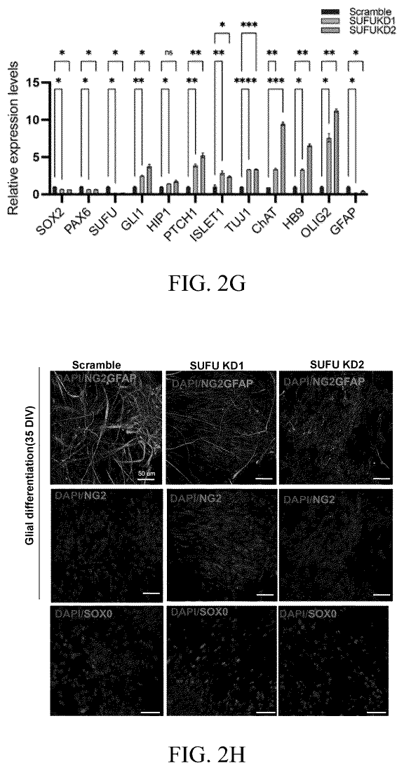

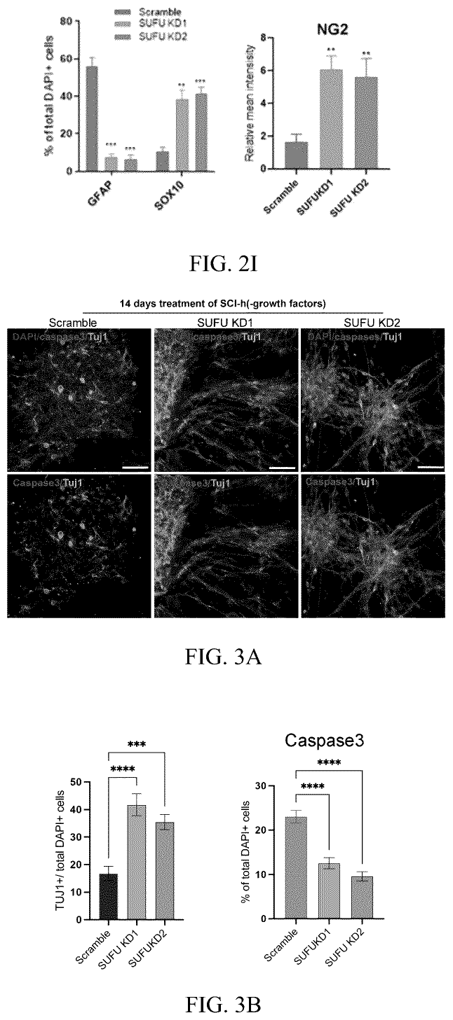

Cell intrinsic activation of SHH in SUFU-KD hNPCs leads to a further investigation of the potential of these modified NPCs in regulating axonal projections/outgrowth, synapse formation, motoneuronal maturation, and oligodendrocytes differentiation, especially under the condition lacking supplementing growth signaling (e.g., EGF, FGF, GDNF, BDNF, and IGF) that is typically deficient in the injured spinal cord. Neurospheres (passage <5) derived from scramble and SUFU-KD group are cultured in well-coated plates without adding neurotrophic factors (GDNF, BDNF and IGF). After 7 days of culturing, a robust axonal outgrowth, labelled by the neurofilament marker (NF) in SUFU-KD cells, exhibiting an increased number of nerve fibers with much longer extension from the core of neurosphere compared to the scramble control is observed (FIG. 2A and FIG. 2B). In addition, a substantial portion of the SUFU KD cells expresses ISLET1/2 (KD1=43.9±6.3%; KD2=47.98±6.9% versus scramble: 3.9±1.02%) among the MAP2+ pan-neuronal population on 7 days post-differentiation, indicating accelerated motoneuronal differentiation (FIG. 2C and FIG. 2D). At 21 days, more SUFU KD cells express mature motoneuronal marker, choline acetyltransferase (ChAT) and HB9, which are associated with increased expression of the presynaptic marker synaptophysin (Syn) compared to the scramble control (FIG. 2E and FIG. 2F). Consistently, qPCR analysis further confirms significant downregulation of neural genes (SOX2 and PAX6) and upregulation of motoneuronal-associated genes (ISLET1/2, HB9, ChAT), ventral gene (NKX6.1) and pan neuronal gene (TUJ1) upon SUFU-KD (FIG. 2G). These results suggest that knockdown SUFU in hNPCs enhances cell intrinsic differentiation potency, promoting neurogenesis and maturation bias toward motor neuron fate in the absence of extrinsic neurotrophic factors support.

Next, the glial differentiation capacity, including astrocytes and oligodendrocytes, in SUFU-KD hNPCs is evaluated. After 35 days of culturing, GFAP+ astrocytes are more frequently observed in the Scramble hNPCs group compared to SUFU-KD groups (FIG. 2H and FIG. 2I). In contrast, the SUFU-KD group shows a significantly increased cell population expressing oligodendrocyte markers SOX10 and NG2 compared to scramble (FIG. 2H and FIG. 2I), most likely due to increased OLIG2 progenitors induced by SHH activation (FIG. 1H). Together, these results suggest that inhibition of SUFU in hNPCs favors the formation of therapeutic cell types for SCI, including motorneurons and oligodendrocytes.

### Example 3. Non-Cell Autonomous Effects of SUFU-KD hNPCs on Cell Survival and Differentiation

SHH is neuroprotective and functioned as a survival-promoting factor for tissue repairing. To determine whether SUFU-KD hNPCs with cell intrinsic SHH activation counters the adverse effects from the injured spinal cord niche, hNPCs from Scramble and SUFU-KD are cultured in the absence of neurotrophic factors and treated with cleared homogenate (100 μg/ml) from the injured spinal cord (SCI-H) for 2 weeks. The spinal cord homogenate is prepared by the following procedure. In brief, rats are perfused with ice-cold saline two weeks after injury, and around 5 mm long segment of the injured spinal cord centered on the injury epicenter is collected and rapidly frozen on in liquid nitrogen. Spinal cords from at least 4 SCI rats are pooled together and is grind to a fine powder under liquid nitrogen by using pestles, while kept on ice. The homogenate is suspended in DMEM/F12 and cleared by centrifugation at 12000 g for 10 min at 4° C. The total protein concentration of cleared supernatants is measured using a BCA test. After adjusting the total protein concentration, the aliquots are kept at −80° C. until use.

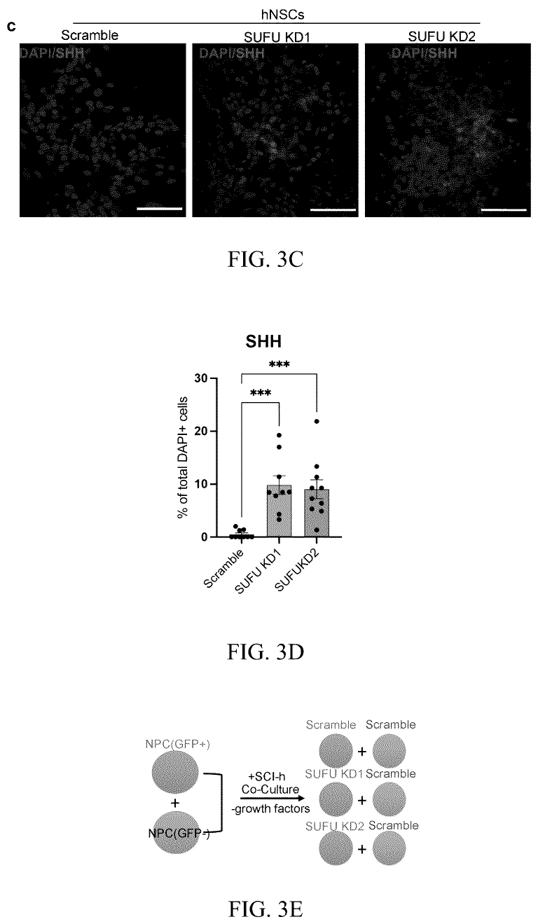

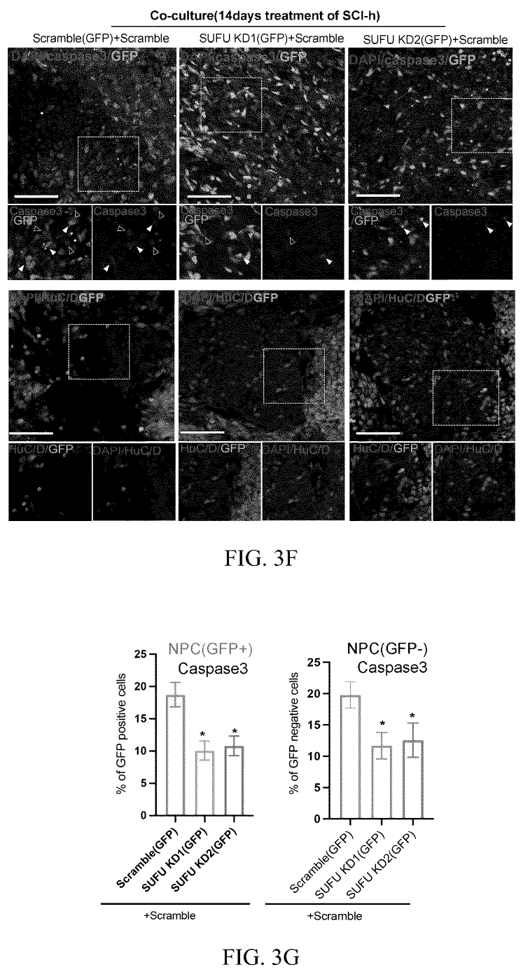

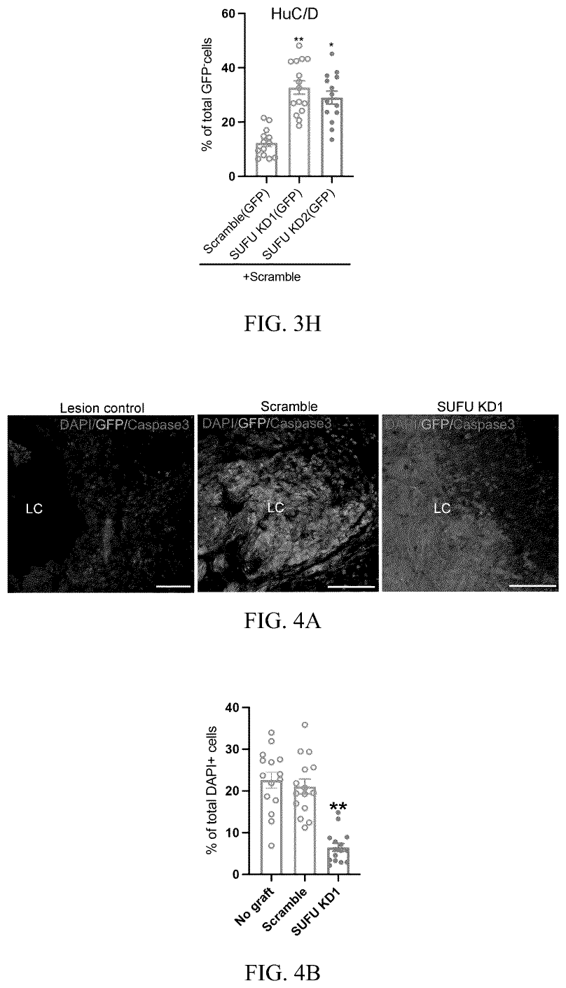

The results show that only a small portion of scramble cells differentiated into TUJ1+ neurons (16.72±2.67%) with few axonal outgrowths when exposed to homogenate (SCI-H). Additionally, a significant increase is observed in apoptosis marked by caspases 3 in scramble cells treated with SCI-H after 2 weeks of culture (FIG. 3A and FIG. 3B). In contrast, SUFU-KD cells mitigated this effect, generating a substantial amount of neurons (41.68±4.04% in SUFU-KD1; 35.42±2.76% in SUFU-KD2) with thick nerve fibres and exhibiting a low percentage of caspases 3 expressions as compared to scramble cells upon treatment of SCI-H (FIG. 3A and FIG. 3B). It is interesting to note that SUFU-KD cells not only show elevated SHH signaling effectors intrinsically but also express increased SHH protein production in culture, implicating the non-cell autonomous effects via SHH exposure (FIGS. 3C and 3D). To examine the ability of SUFU-KD hNPCs to modulate other cell activities, SUFU-KD (GFP labelling) and scramble (GFP or Non-GFP labelling) are co-cultured (FIG. 3E). Non-GFP scramble NPCs co-cultured with GFP scramble NPCs or SUFU-KD GFP NPCs are treated with cleared homogenate (100 μg/ml) without supplementing neurotrophic factors. The non-GFP scramble cells cultured with GFP-scramble cells exhibit increased apoptosis with few differentiating neurons (HuC/D+) after 14 days of culturing (FIGS. 3F-3H). Conversely, scramble (non-GFP) cells cultured with SUFU-KD (GFP+) hNPCs exhibit a significantly lower percentage of apoptosis and are able to form more HuC/D+ neurons after 14 days culturing (FIGS. 3F-3H). All these data demonstrates that the non-cell autonomous effects of SUFU-KD NPCs in regulating cell survival and differentiation.

### Example 4. SUFU-KD NPCs Modulate the Injured Niche for Survival and Connection

After confirming the superiority of SUFU-KD NPCs in vitro, it is further examined to see if SUFU-KD NPCs offer a beneficial effect for the injured spinal cord. Thoracic contusion injury is induced at level T8 in rats, followed by cell transplantation at 2 weeks post-injury. Briefly, Sprague-Dawley male rats are anesthetized with an intraperitoneal injection of a ketamine (80 mg/kg) and xylazine (10 mg/kg) mixture. A laminectomy is conducted at the caudal portion of T6 and at all T7 spinal levels. A T8 severe contusion SCI (weight of 25 g, height of 50 mm) is produced with a modified version of MASCIS impactor. After performing the spinal contusion, muscle and skin layers were sutured with 4.0 polyglactin. The bladder of each injured animal was squeezed manually twice a day after SCI for 2 to 3 weeks. Cyclosporine is applied daily to prevent immune rejections for human grafts in rats. Fourteen days after SCI surgery, animals undergo a second procedure for cell implantation. A total of 2×10⁵ GFP-expressing scramble control or SUFU-KD hNPCs are grafted into the lesion site (1 μL administered into the left and right sides of the injury site) without growth factors supplementation. The rats are anesthetized with an intraperitoneal injection of a ketamine (80 mg/kg) and xylazine (10 mg/kg) mixture. The original incision is reopened, and the injury sites are re-exposed. To investigate the survival and spontaneous differentiation capacity of scramble and SUFU-KD hNPCs, Scramble and SUFU-KD hNPCs are resuspended in DMEM/F12 medium supplemented with 10 μM Rock inhibitor at a density of 10⁵ cells/μL without growth factors. Cells are kept on ice throughout the procedure. Two injections are performed at the injury site, each delivering 2.0 μL of the cell suspension at −0.5 and +0.5 mm distance from the dorsal middle line (0 mm). The injection is performed using a 30G syringe (Hamilton) connected with a micropump (RWD), with the animals tightly fixed in a stereotaxic apparatus (RWD). The injection rate is 250 nL/min. The syringe is left in place for an additional 10 min before and after the injections. At the end of the procedure, the muscle and skin layers are sutured with 4.0 polyglactin, and the animals receive subcutaneous injections of buprenorphine (0.03 mg/kg) and meloxicam (2 mg/kg) for 3 days, and oral administration of enrofloxacin (2.5%) for 7 days. Cyclosporine (5 mg/kg) is subcutaneously injected every day for immune-suppression. Animals undergo functional testing for up to 12 weeks and are sacrificed for anatomical analysis by transcardial perfusion with 4% formaldehyde.

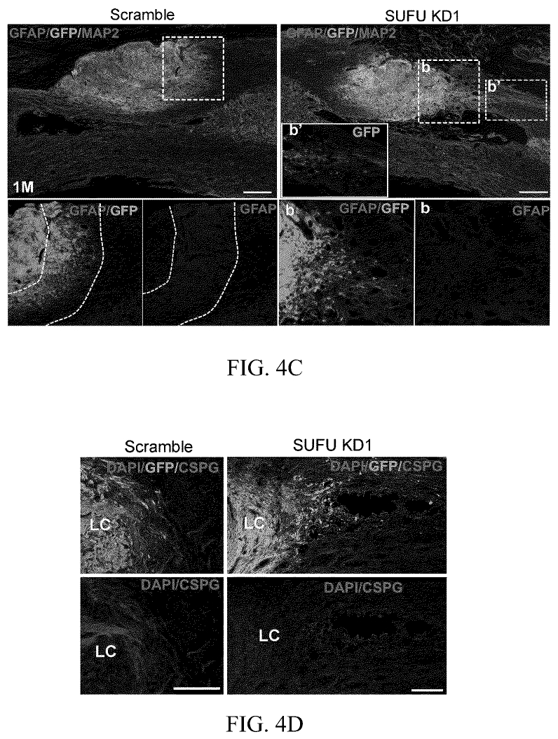

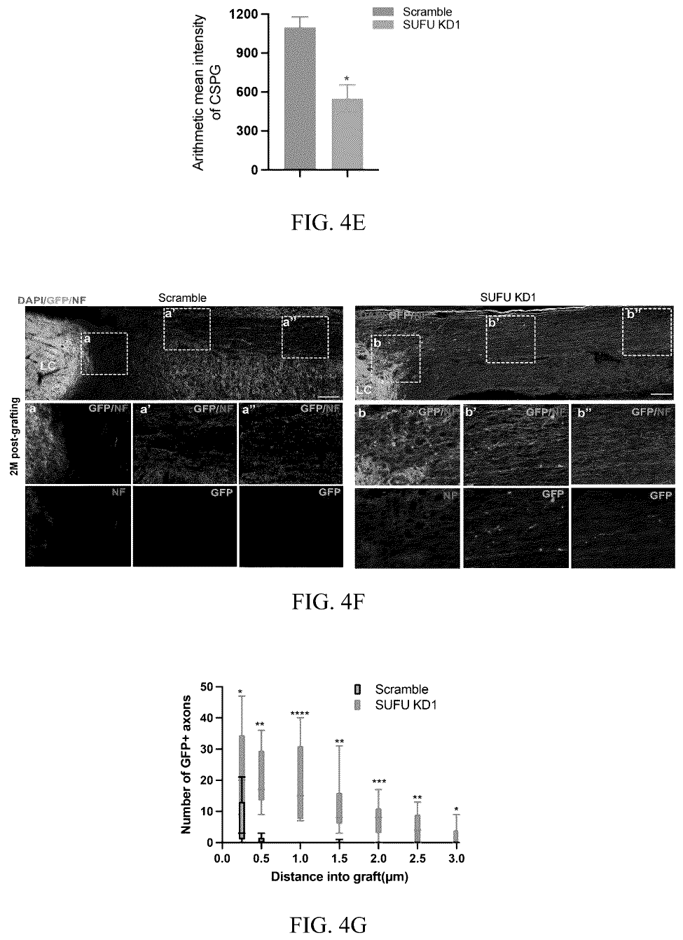

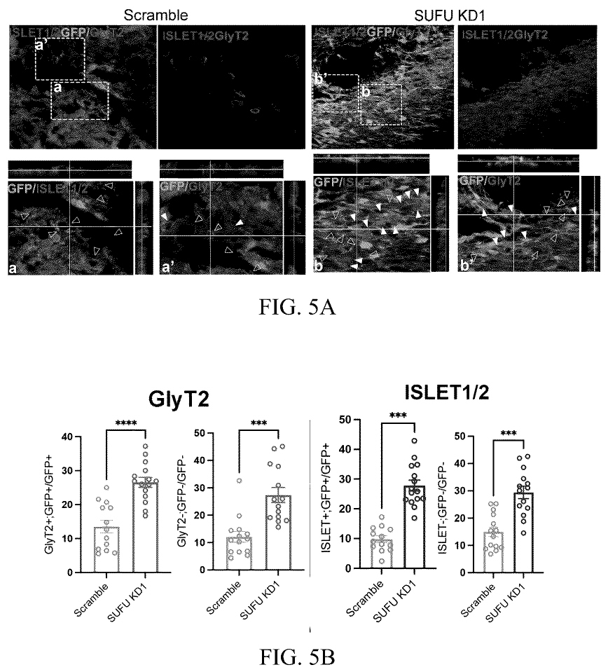

Anatomical analysis shows that both scramble and SUFU-KD hNPCs expand and survive in grafted animals (FIG. 4A), suggesting successful integration of host tissues in the lesion site and compensation for the dramatic tissue loss within the cavity. For both lesion control and Scramble group, a substantial amount of caspase 3+ apoptotic cells are found in the periphery of the lesion epicenter or within the grafts, whereas SUFU-KD grafts significantly reduce caspases 3 expressions around lesion sites at 1M post-grafting (FIG. 4A and FIG. 4B). The lesion site in the injured spinal cord is surrounded by glial scar with robust expressions of GFAP, prohibiting nerve extension and reconnection from scramble grafts. Interestingly, GFAP+ glial barrier is relatively less condensed in the injured spinal cord grafted with SUFU-KD NPCs, which exhibit massive axonal branches that penetrated the GFAP-expressing glial scar as early as 1 month post-graft (1M) (FIG. 4C).

To determine whether SUFU-KD grafts facilitate nerve outgrowth by modulating glial scar, the expression of CSPGs in the injured spinal cord is further examined. CSPGs are produced by reactive astrocytes and act as a key inhibitory component to limit axonal outgrowth and regeneration, oligodendrocyte replacement, and remyelination 28. In agreement with the literature, upregulated CSPG expression forms a complex barrier around the lesion sites in response to the injury, restricting the nerve regeneration and outgrowth of scramble grafts (FIG. 4D and FIG. 4E). In contrast, SUFU-KD graft significantly inhibits CSPG deposition, which displays more dispersed expressions around lesion sites, leading to a number of GFP+ axonal outgrowth from the grafts for reconstituting neuronal connections (FIG. 4D and FIG. 4E). At 2 months post-grafting, a robustly projected neurofilament (NF+) derived from SUFU-KD graft is detected, whereas scramble grafts are restricted within the lesion sites without obvious axonal outgrowth across the lesion site (FIG. 4F). Importantly, a large number of GFP+ axons co-expressing NF emerging from SUFU-KD grafts extends into the injured spinal cord and extending much further caudally by more than 25 mm at 2 months post-graft (FIG. 4F and FIG. 4G), recapitulating the long nerve fibers extension observed in vitro. These results indicate that the transplanted SUFU-KD hNPCs exert non-cell autonomous effects, modulating the injured niche to enhance the survival and the reconstitution of neuronal connections.

### Example 5. SUFU-KD Grafts Promote Robust Neurogenesis Intrinsically and Extrinsically in the Injured Spinal Cord

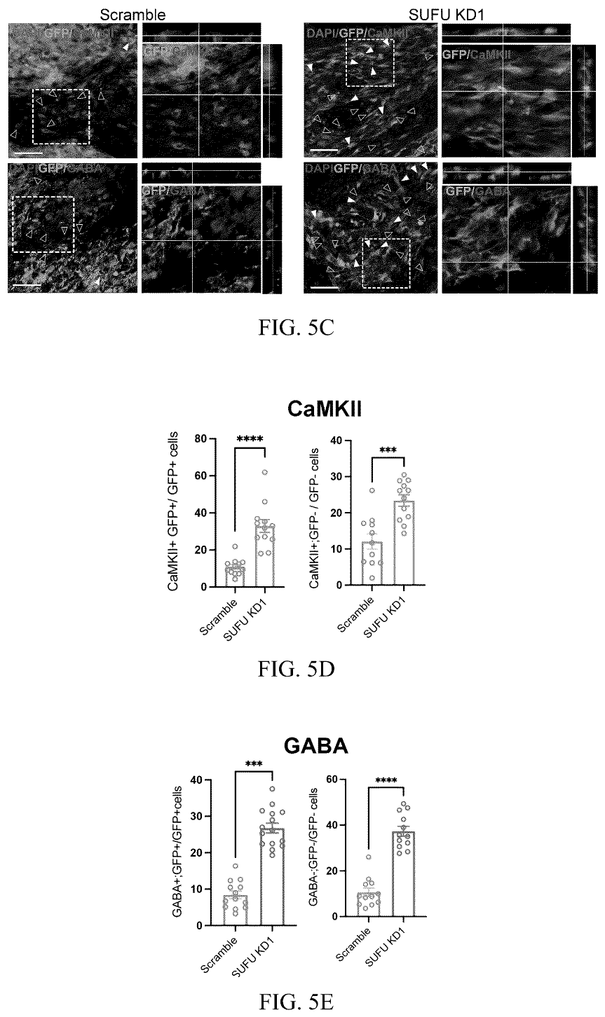

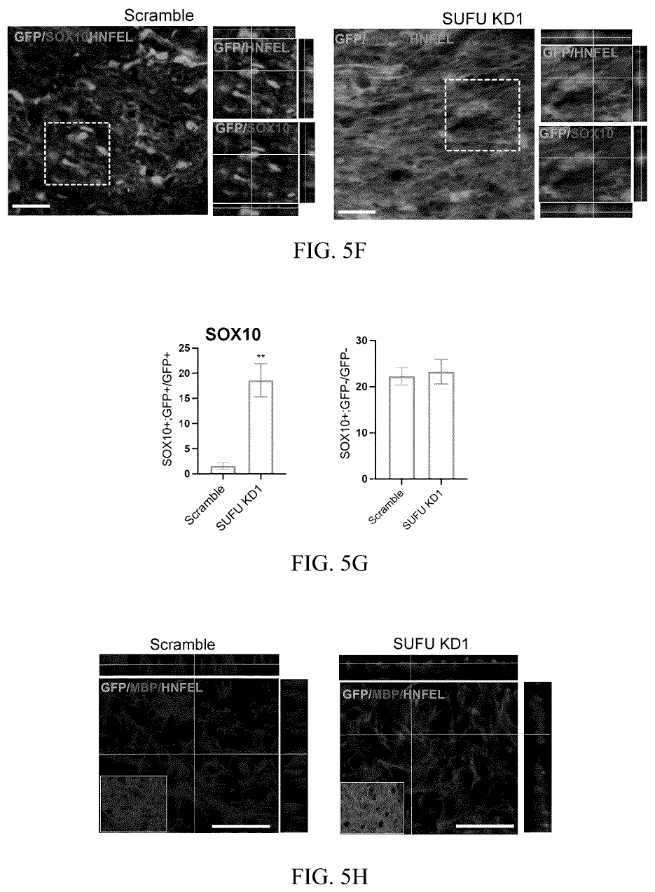

The regenerative properties of scramble and SUFU-KD hNPCs grafts are further evaluated to determine the ability of grafted SUFU-KD hNPCs to surmount the injury environment that lacks growth factors and generate therapeutic cell types. As shown in FIG. 5A to 5E, scramble grafts give rise to fewer mature neurons while SUFU-KD grafts generate a much higher proportion of mature neurons, including motoneurons (ISL1/2, SUFU-KD: 27.8±4.05% versus Scramble: 9.78±4.1%), glycinergic inhibitory neurons (SUFU-KD: 26.53±5.74% versus Scramble: 13.54±6.87%), CaMKII+ excitatory neurons (SUFU-KD: 32.93±12.02% versus Scramble: 10.88±4.52%) and GABA+ neurons (SUFU-KD: 26.76±5.26% versus Scramble: 8.41±3.87%). Strikingly, a significantly increase of non-GFP host cells is observed around SUFU grafts expressing mature neuronal markers compared to non-GFP cells from scramble grafts, including glycinergic inhibitory neurons (SUFU-KD: 27.36±10.23% versus Scramble: 12.07±7.30%), CaMKII+ excitatory neurons (SUFU-KD: 23.40±5.49% versus Scramble: 12.10±7.01%) and GABA+ neurons (SUFU-KD: 37.30±7.58% versus Scramble: 10.64±6.33%) ISLET1/2 (FIG. 5A-5E). All these data suggest that SUFU-KD grafts not only exhibit enhanced neurogenic potential intrinsically but also exert beneficial effects on the injured environment by promoting neurogenesis of host cells.

To further investigate whether SUFU grafts promote oligodendrocytes in the injured spinal cord, the markers of SOX10 and myelin basic protein (MBP) for oligodendrocytes in grafts are also examined. As shown in FIGS. 5F and 5G, it is found that a substantial amount of SOX10 expression is detected in SUFU-KD grafts, whereas SOX10 expression is barely detectable in the scramble grafts (SUFU-KD: 18.61±4.23% versus Scramble: 1.60±1.81%). The percentage of non-GFP host cells expressing SOX10 is comparable in both groups, suggesting SUFU grafts do not exert non-cell autonomous effects on regulating oligodendrocytes properties. Consequently, SUFU-KD-derived axons are myelinated, as evidenced by the co-localization of MBP-labelled myelin sheath with HNEFL+ axons, whereas no MBP expression is detected with axons emerging from the scramble graft (FIG. 5H). Altogether, these results demonstrate that SUFU-KD hNPCs have intrinsic and extrinsic superiorities in generating therapeutic cell types and modulating injury niche for the treatment of SCI.

### Example 6. SUFU-KD Grafts Effectively Establish the Integration into Host Neural Circuits

The effectiveness of grafts in integrating with the host neural circuitry and promoting tissue repair for locomotion recovery is evaluated by injecting AAV1-CMV-mCherry into motor cortex for antegrade tracing of trans-synaptic connectivity after injury. Briefly, three weeks before sacrifice, the skulls of anesthetized rats (ketamine (80 mg/kg) and xylazine (10 mg/kg) are tightly fixed to a stereotaxic apparatus (RWD). AAV2/1-hSyn-mCherry (brainVTA, 2.5×10¹² vg/mL) is injected into five spots of the right motor cortex. A vertical midline incision is made from between the eyes to the posterior skull. The injection area on the right hemisphere is defined in a rectangle measuring 2 mm (from 1.0 mm anterior to −1.0 mm posterior to the bregma) by 1.5 mm (lateral to the bregma). A drill is used to create the injection sites on the skull. Injections are performed using a 33 G syringe attached to a micropump. Each injection delivers 0.5 μL of the virus solution into the motor cortex at a rate of 100 nL/min. The injector tip is left in place for additional 5 min before and after the injections. Animals are euthanized 3 weeks following injection and postsynaptic structures are examined for the presence of cell body labeling.

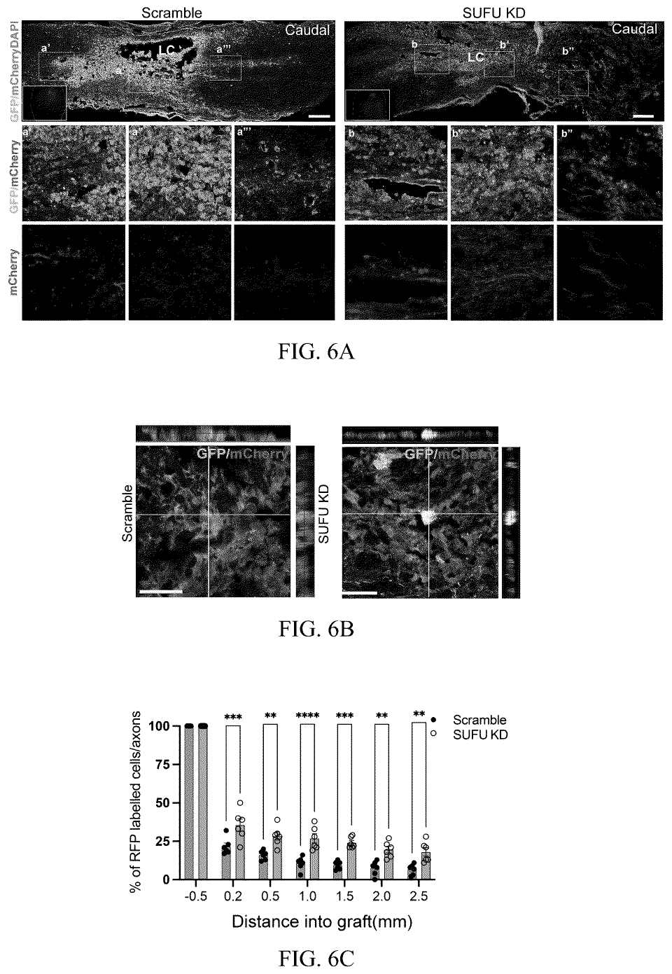

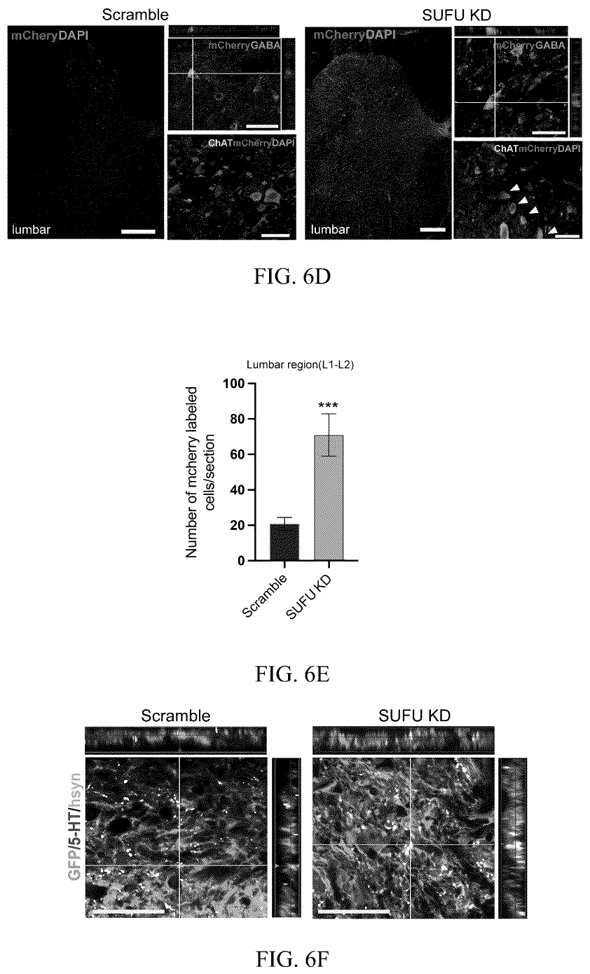

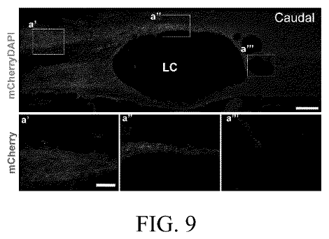

As shown in FIG. 6A, the histological analysis shows a strong mCherry+ signal in the brain injection sites and injury site rostrally in lesion control, scramble and SUFU-KD graft groups at 2 weeks post-injections (FIG. 9). In the lesion control group, trans-synaptic transmission is severely blocked by the cavity in the injured spina cord, resulting in absence of mCherry+ cells in the caudal region of the lesion sites, confirming disrupted neural circuits following contusive SCI (FIG. 9).

For quantification of neural differentiation or growth in human cell grafts, eight to nine randomly selected fields of grafts from six to eight animals per group are visualized using a confocal microscope at a magnification of 100× or 200×. The number of mCherry-expressing axons regenerating into grafts in the lesion sites is quantified. In brief, dorsal-to-ventral virtual lines of one in six sections (30 μm thickness) are placed at regular distances under 100× magnification, and graft/host interface and the number of axons intercepting labelled for 5-HT or mCherry-labeled axons are examined and counted under 200× magnification. In the scramble NPCs group, only a few mCherry+ cells are observed in the lesion sites and caudal to lesion site, indicating less effective trans-synaptic transmission. In contrast, strong mCherry signals are detected in the lesion sites overlapping with GFP+ cells and several millimetres caudal to the lesion site with SUFU-KD graft, confirming trans-synaptic spread of mCherry from host neurons to the graft (FIG. 6A-6C). In the lumbar spinal cord, a larger number of mCherry+ cells are present in ventral and dorsal horns of the spinal cord grafted with SUFU-KD NPCs, co-localized with ChAT+ motoneurons and GABA+ sensory interneurons, representing long descending propriospinal neurons in the spinal cord (FIGS. 6D and 6E). These observations indicate that SUFU-KD grafts effectively integrate with both long-projecting and local spinal cord circuitries. In addition, some 5-HT-positive nerve fibers grow into the injury sites and form synaptic contacts with SUFU grafts, whereas few synaptic connections are established between 5-HT-positive nerve fibers and in the area caudal lesion site of scramble graft (FIG. 6F). These results demonstrate that SUFU graft promotes the re-establishment of synaptic connectivity with the major host neural circuitry that normally project to the spinal cord effectively.

### Example 7. SUFU-KD Grafts Improve Hindlimb Function after Contusive SCI

To evaluate the effect of scramble and SUFU-KD grafts on functional recovery after SCI, a series of motor function tests is performed during the 16-weeks post-injury period. Hindlimb locomotor activity in the lesion control, receipts with scramble and SUFU-KD grafts are assessed weekly by the Basso, Beattie, and Bresnahan (BBB) locomotor scale, starting 7 days before and after injury. The BBB open-field 21-point locomotion rating scale is used in the weekly assessments of rats conducted by two independent observers. Grid walk assessment is performed using a modified grid (4×6 cm grids), and hind limb foot drops are recorded as a measure of hind limb sensorimotor function. Two investigators are blinded to group identity assessed outcomes. Foot drops are recorded if the rat is unable grasp a grid rung with a hind paw during stepping and paw placement, resulting in a foot drop below the grid. The percentage of total paw replacements=Total steps-foot drops/Total steps (including left and right hind limb). Testing is performed on uninjured rats prior to surgery as a baseline measurement, and then every 2 to 3 weeks post-injury. Three trials per rat are evaluated and the scores are averaged for the analysis. For the foot fault scoring, the score is defined as follows: correct placement=6 points; partial placement=5 points; placement correction=4 points; replacement=3 points; slight slip=2 points; deep slip=1 point; and total miss=0 points. With the limb that starts the walk, consecutive steps are then estimated. Three trials per rat are evaluated and the scores are averaged for the analysis. For the footprint analysis, animals are placed on 1 meter long narrow corridor lined with force sensors that recorded the pressure exerted by the feet due to locomotor behavior, which is converted into digital image of plantar surface, which reflects the hind limb supporting ability and coordination. Rats are then allowed to run three times along the corridor, and stereotyped gait and motor coordination parameters, including hind limb stride length and width are measured from three complete step cycles from the middle of the runway.

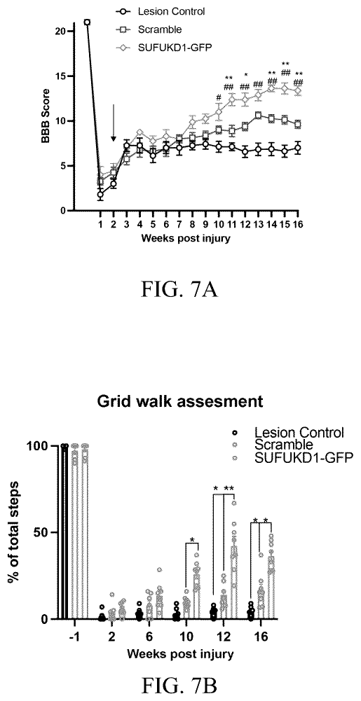

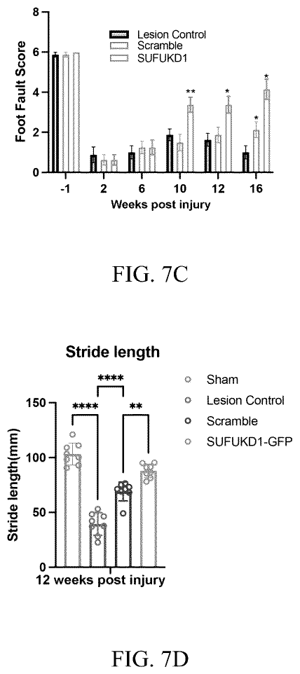

At 1- and 2-weeks post-injury, all treatment groups show substantial loss of locomotor function, with an average BBB score less than 5 (little or no hindlimb movement). Scramble recipients start to show improved locomotor function at 12 weeks (10-weeks post-graft) compared to lesion control, whereas the SUFU-KD recipients exhibit significant improvement in hindlimb motor function at 10 weeks (8-weeks post-graft) to the end of the experiment (16-weeks post-injury) compared to lesion control (FIG. 7A). In addition, SUFU-KD recipients show much better improvement in hindlimb motor function compared to scramble recipient animals at 11-weeks post-injury (FIG. 7A).

To further evaluate skilled locomotor function and coordination, the grid-walking test is employed by counting the percentage of correct steps out of paw replacements and foot faults as rats traverse the metal grid. After 2-weeks post-injury (before grafting, lesion control), rats lose the ability to place their hind paw correctly on the metal grid. The SUFU-KD grafted rats exhibit much better performance in placing their affected hind paw (left and right) correctly on the grid with less misdirected steps compared to the scramble recipients from 10-weeks post-injury, which show a mild improvement by week 12 compared to lesion control (FIGS. 7B and 7C). Subsequently, the gait of the transplanted animals is assessed by the footprint test, in which the pressure exerted by the feet during locomotor activity is converted into a digital image of the plantar surface by a force sensor, indicating limb stepping ability and coordination. Quantitative analysis of stride length in grafted animals further confirms a remarkable improvement in SUFU-KD-grafted animals with values higher than the scramble and lesion controls (FIG. 7D). These results demonstrate the enhanced therapeutic potential of SUFU-KD grafts for the restoration of locomotor function in a rodent model of contusion SCI.

Overall, it is demonstrated that hNPCs with SUFU inhibition activate SHH signaling, promoting survival and intrinsic differentiation in generating therapeutic cell type without reliance on trophic factors support. Strikingly, these modified NPCs also exert non-cell autonomous effects, which facilitate endogenous neurogenesis and modulates microenvironment by reducing progressive apoptosis and CSPG+ suppressive barriers around lesion sites. The combinational beneficial effects of SUFU inhibition hNPCs greatly improves locomotor functions in a severe traumatic injury model.

The functional recovery achieved by hNPCs graft is largely determined by survival, cell replacement and integration into the host tissue. Notably, SUFU-KD grafts are more resilient to the detrimental environment after SCI, showing increased survival and strong neurogenic potency in generating substantial amounts of mature neuronal subtypes compared to scramble grafts in the absence of trophic support. These observations suggest that the repression of SUFU in progenitors from different regions of the CNS and PNS is required for the onset of neuronal or glial differentiation. It is well-known that Sox10 directs NPCs toward the oligodendrocyte lineage by decreasing SUFU expressions. However, the modified hNPCs with SUFU inhibition also promote robust formation of oligodendrocytes marked by SOX10 and NG2, which facilitate the myelination of axons outgrowth and contributed to the locomotion recovery following the SCI. In addition, matched grafts are able to rewire the lesioned spinal cord. Accordingly, SUFU-KD grafts reduce apoptosis and formation of inhibitory astrocytic scar expressing CSPGs around the lesion cavity, which enable long-distance axonal growth into the caudal recipient spinal cord beyond the lesion site. Interestingly, SUFU grafts also promote neuronal differentiation and maturation of non-modified hNPCs and host cells, as a significant increase of subtypes of neuronal markers is consistently observed around SUFU-KD NPCs in vitro and in vivo. The striking differences in engraftment between scramble and SUFU-KD hNPCs may be due to the upregulated expression of SHH ligand, which likely acts as a trophic factor to promote the survival, proliferation, and neuronal differentiation of non-modified hNPCs and endogenous host cells.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method for treating spinal cord injury, comprising:
administering a genetically engineered neural progenitor cell (NPC), wherein the genetically engineered NPC's genome is genetically modified for suppressing a negative regulator of sonic hedgehog (SHH) signaling pathway, so as to upregulate the SHH signaling pathway.

2. The method of claim 1, wherein the upregulation of SHH signaling pathway is achieved by suppressing the negative regulator of SHH signaling pathway.

3. The method of claim 2, wherein the negative regulator is suppressor of fused homolog (SUFU).

4. The method of claim 1, wherein the administration is grafting the genetically engineered NPC directly into a lesion site of a subject in need thereof.

5. The method of claim 1, wherein the upregulated SHH signaling pathway reduces apoptosis and enhances the survival and differentiation of host neurons at the lesion site.

6. The method of claim 1, wherein the genetically engineered NPC reduces suppressive barriers derived from glial scar around the lesion sites.

7. The method of claim 1, wherein the administration of the genetically engineered NPC is combined with a physical therapy regimen to enhance functional recovery.

8. A composition comprising a genetically engineered NPC for treating spinal cord injury in a subject in need thereof, further comprising a pharmaceutically acceptable addition, wherein the genetically engineered NPC's SHH signaling pathway is upregulated.

9. The composition of claim 7, wherein the upregulation of SHH signaling pathway is achieved by suppressing a negative regulator of SHH signaling pathway.

10. The composition of claim 8, wherein the negative regulator is SUFU.

11. The composition of claim 7, wherein the pharmaceutically acceptable addition comprises an excipient, a stability additive, a carrier, a diluent, and a solubilizer.

12. The composition of claim 10, wherein the pharmaceutically acceptable additive further comprises a neurotrophic factor.

13. The composition of claim 7, the composition further comprises a sustained release system to provide prolonged exposure of the genetically engineered NPC at the lesion site.

14. The composition of claim 7, wherein the composition is formulated for direct delivery to a lesion site of the subject.

15. The composition of claim 7, wherein the composition is formulated as an injectable gel or scaffold or cell suspension for controlled delivery to the lesion site.

16. Use of a composition comprising a genetically engineered NPC for treating spinal cord injury in a subject in need thereof, wherein the genetically engineered NPC's SHH signaling pathway is upregulated.

17. The use of claim 15, wherein the upregulation of SHH signaling pathway is achieved by suppressing a negative regulator of the SHH signaling pathway.

18. The use of claim 16, wherein the negative regulator is SUFU.

19. The use of claim 15, wherein the genetically engineered NPC's upregulated SHH signaling pathway promotes remyelination of damaged/regenerated axons.


SEQUENCE LISTING (2 sequences)

SEQ ID NO: 1 - DNA, 21 bp, Homo sapiens
  source 1..21: mol_type = genomic DNA
  gene 1..21: gene = partial of Human SUFU gene
aacagagtccatgagtttaca

SEQ ID NO: 2 - DNA, 21 bp, Homo sapiens
  source 1..21: mol_type = genomic DNA
  gene 1..21: gene = partial of Human SUFU gene
aacagtgagtcaagaattcag